(19) United States
(12) Patent Application Publication
(10) Pub. No.: US 2003/0133893 A1
(43) Pub. Date: Jul. 17, 2003

(54) Compositions and methods for treating skin ailments

(75) Inventors: Moire Marx Nir, Misgav (IL); Irina Elisyevich, Katzrin (IL); Omri Mairon, Zichron Yaakov (IL); Oded Stein, Jerusalem (IL)

Correspondence Address: G.E. EHRLICH (1995) LTD., c/o ANTHONY CASTORIN, SUITE 207, 2001 JEFFERSON DAVIS HIGHWAY, ARLINGTON, VA, 22202, US

(73) Assignee: Degania Silicone Ltd.

(21) Appl. No.: 10/044,941

(22) Filed: Jan. 15, 2002

(51) Int. Cl. A61K 7/06; A61K 7/11

(52) U.S. Cl. 424/70.11

(57) ABSTRACT

Compositions that comprise a polymer entrapping an oxidizing agent are disclosed. The disclosed compositions are used in the treatment of skin ailments such as human papilloma virus infections.

### FIELD AND BACKGROUND OF THE INVENTION

The present invention relates to compositions and methods for treating skin ailments and, more particularly, to the use of a conformable and/or spreadable polymer as a sustained release carrier for an oxidizing agent entrapped therein or thereby and its use in the treatment of skin ailments such as, but not limited to, human papilloma virus infections.

Human papilloma virus (HPV) infections are common infections of the outer layer of the skin, which affect most persons sometime during their lifetime. To date, more than sixty types of HPVs have been identified.

HPV infections typically emerge as skin warts, which include, for example, common warts (verruca vulgaris), plantar warts, palmar warts, planar warts (verruca plana), mosaic warts, and venereal warts (condyloma accuminatum). These skin growths are unsightly and irritating, and although the majority of such infections are benign and self-limited, there are subtypes of papilloma virus that are considered pre-malignant in certain clinical settings. Therefore, the removal of emerged skin warts is highly recommended.

Throughout the years a number of therapies have been developed for treating these coetaneous infections. However, most of the presently known methods of treating warts are painful, expensive, time consuming or ineffective.

The presently used methods of treating HPV infections typically include the use of locally destructive chemicals or agents, such as salicylic acid, lactic acid, trichloracetic acid, dichloroacetic acid, nitric acid and glacial acetic acid; surgically destructive methods such as excision, electrocautery, electrodesiccation, curettage, blunt dissection and laser vaporization or coagulation; blister-producing methods such as liquid nitrogen cryotherapy, carbon dioxide cryotherapy and cantharidin; cellular inhibition, which uses agents such as podophyllin and podophyllotoxin, 5-fluorouracil, bleomycin, colchicine, interferon local injections and radiation; altering the cutaneous environment, which includes agents or techniques such as retinoids, formalin, glutaraldehyde, aluminum chloride and heat therapy; and immune stimulation methods of treatment, which include dinitrochlorobenzene (DNCB), interferon systemic injections and vaccination, either autologous or intralesional.

Hence, the presently most common methods of warts treatment can be divided into chemical and physical methods. The physical methods typically include destruction of the infected keratinocytes by cooling (e.g., liquid nitrogen) or heating (e.g., electrocautery, CO₂ laser), and often lead to injury of surrounding tissues, secondary infections and other undesired consequences. The chemical methods commonly use locally destructive chemicals and typically include caustic chemicals that act through nonspecific destructive mechanisms to cause cell death, killing the infected keratinocytes. The keratinocytes are subsequently desquamated from the skin surface. This non-specific form of destructive therapy often causes side effects such as pain, secondary infection, permanent scarring and recurrence.

The chemical methods typically involve topical application of the chemical agents as solutions, tinctures, creams, ointments, patches, etc. One of the presently most used chemical agent for treating warts is salicylic acid, which is typically administered as a patch or a gel. A typical salicylic acid patch contains a quantity of salicylic acid in a sticky base or a rubber base. The salicylic acid/base composition is retained and carried by a piece of adhesive tape. In use, the salicylic acid/sticky base is applied to the wart, and free ends of the adhesive tape are applied to peripheral skin areas to keep the treating agent in place over the skin. A typical salicylic acid gel application includes direct application to the wart and subsequent evaporation of the solvents, which leaves a film with the therapeutic agent. However, the use of salicylic acid in the treatment of HPV requires repeated administration of the composition for a prolonged period of about 4-6 weeks, and is oftentimes unsuccessful.

The presently used methods of topical application of chemical agents for treating warts further include other compositions. For example, U.S. Pat. No. 5,846,559 discloses a skin patch for the delivery of a contactant to human skin, which induces cell-mediated contact dermatitis. The disclosed skin patch preferably includes a polymeric gel matrix, which enables a controlled release of the contactant to the skin upon application. The contactants described in this patent include dinitrochlorobenzene, diphenylcyclopropenone, squaric acid dibutyl ester and their derivatives.

U.S. Pat. No. 5,476,664 discloses a method of treating warts which includes repeated application of an adhesive tape that contains an anthralin active ingredient and a pharmaceutical carrier. The adhesive tape adheres to peripheral skin areas and provides an occluding environment for the wart treated with the composition.

U.S. Pat. No. 5,576,716 discloses traumatic acid salts and compositions containing same that can be used in the treatment of cutaneous pathology. The disclosed traumatic acid salts have antibiotic, antiseptic, disinfectant, antifungal and antiviral activity. However, the disclosed traumatic salts are preferably used to promote reepithelialization and are not specifically active with respect to HPV. The traumatic acid salts compositions of this patent are administered, parenterally or topically, as aqueous solutions and are therefore not suitable for continued application, which is recommended for HPV skin warts.

An effective antimicrobial agent, which has a wide antibiocidial activity (e.g., antibacterial, antifungal and antiviral activities) is “free chlorine”. “Free chlorine”, a phrase which is used herein to describe [Cl⁺], is routinely used in water treatment systems [Handbook of Chlorination and Alternative Disinfectants, 4^{th} Ed., G. C. White, Wiley, 1998]. Hypochlorous acid, HOCl, is a common source of free chlorine and is typically used as an aggressive oxidizing and chlorinating agent. HOCl is commonly used in water purification systems.

The total mechanism of the anti-biocide action exerted by free chlorine is not yet understood and is assumed to depend on the type of the attacked entity (e.g., vegetative bacteria, spore, virus, or fungus). However, the ability of HOCl to penetrate the microorganism's cell wall appears to be attributed both to the structure and size of HOCl, which are similar to that of water, and to its electrical neutrality [Handbook of Chlorination and Alternative Disinfectants, 4^{th} Ed., G. C. White, Wiley, 1998].

Direct use of HOCl is limited due to its high activity and aggressiveness as an oxidizing agent. Hence, the use of compounds that are capable of releasing HOCl and thus act as indirect oxidizing agents is preferred.

Chlorinated isocyanurates, such as the commercially available trichloro(iso)cyanurate, which is also referred to hereinafter as TCIA, and sodium dichloro(iso)cyanurate, which is also referred to hereinafter as DCIA, are known to produce HOCl upon reaction with water. These reagents are highly active, even at very low concentrations, and are therefore widely used as water disinfectants and bleaching agents.

Nevertheless, although these reagents act as an efficient free chlorine source, and thus can exert wide biological activity at very low concentrations, their use as agents for skin treatment has not been extensively explored. In this respect, Boddie and Nickerson [Boddie, R. L. and Nickerson S. C., J. Dairy Sci., 1996, 79(9), 1683-8] have reported two teat dip sodium dichloro(iso)cyanurate formulations, which release hypochlorous acid (2800 ppm) as the active ingredient, that were found substantially efficient against new Staphylococcus aureus and Streptococcus agalactiae IMI and did not exert adverse effects on teat skin condition. However, no other uses of chlorinated isocyanurates in skin ailment treatment have been studied or disclosed so far.

WO 9965538 discloses anti-infective medical devices that comprise a polymeric matrix with an oxidant producing component trapped within the matrix, which is stable at least until the device is contacted by water. The disclosed medical devices in this application are used as implantable devices such as catheters, living skin matrices or wound dressings, for insertion into various body cavities or over wound, to confer to the site microbicidial or virucidal activity. Nevertheless, WO 9965538 fails to disclose the use of such medical devices in a treatment of skin ailments that require application of spreadable and/or conformable compositions, such as HPV. Furthermore, this application fails to disclose the use of compositions that contain chlorinated isocyanurates.

U.S. Pat. Nos. 5,562,652 and 5,616,119 disclose water vapor-activatable apparatus that includes a permeable base material and a water vapor-activatable medicinal agent that diffuses, as a reaction product, through the permeable material to a patient's body, upon exposure to water vapor. The apparatus described in these patents are used in preventing the onset of infections in medicated polymeric devices such as implanted spermicidal devices and chemotherapeutic delivery systems. However, these patents fail to disclose the design and use of a spreadable and/or conformable composition or a medical device that contain an active agent for the treatment of pre-existing skin ailments such as HPV.

Hence, the prior art teaches various compositions for the treatment of skin ailments such as HPV. The presently most widely used methods employ destructive chemical agents and, in particular, a salicylic acid. The presently known compositions are commonly applied in the form of a liquid solution, a gel or an adhesive tape that includes a mixture thereof with a sticky or rubber base. However, the use of these compositions in the treatment of HPV often requires repeated and prolonged applications, and is often unsuccessful. The prior art further teaches the use of oxidant producing components within polymeric matrices in implantable devices, such as catheters.

The prior art fails to teach compositions that contain oxidizing agents, such as free chlorine, or compounds capable of releasing oxidizing agents, such as chlorinated isocyanurates, for treating HPV and other related existing skin ailments that require continued application of liquid or pasty compositions. Evidently, the prior art fails to teach compositions of such compounds, which further contain a polymeric carrier that can be used for the sustained release thereof. Such compositions could be highly advantageous over the presently known compositions for treating HPV and related skin ailments, since they are known to exert a wide and high biological activity at low concentrations and can therefore serve as successful and efficient composition for the treatment of a variety of skin ailments (e.g., viral, bacterial and fungal skin ailments).

There is thus a widely recognized need for, and it would be highly advantageous to have, novel compositions and methods of treating skin ailments, such as HPV, which include oxidizing agents or compounds capable of releasing oxidizing agents, along with a polymeric slow release carrier.

### SUMMARY OF THE INVENTION

According to one aspect of the present invention there is provided a composition comprising a polymer and an oxidizing agent being entrapped in or by the polymer.

According to further features in preferred embodiments of the invention described below, the composition further comprises one or more additive(s) selected from the group consisting of a filler, a salt, a sugar, a glycerin and a glycol.

According to another aspect of the present invention there is provided a pharmaceutical composition comprising, as an active ingredient, an oxidizing agent being entrapped in or by a pharmaceutical sustained-release carrier, the carrier comprises a polymer.

According to further features in preferred embodiments of the invention described below, the pharmaceutical composition is packaged and identified for the treatment of a skin or mucosal membranes ailment.

According to yet another aspect of the present invention there is provided a medical device designed and shaped for applying onto a skin of a subject in need thereof, the device comprising a pharmaceutical composition, which comprises, as an active ingredient, an oxidizing agent being entrapped in or by a pharmaceutical sustained-release carrier, the carrier comprises a biocompatible polymer.

According to further features in preferred embodiments of the invention described below, the medical device has a flat configuration.

According to still further features in the described preferred embodiments the medical device further comprises a backing for backing the pharmaceutical composition when applied.

According to still further features in the described preferred embodiments the medical device is configured as a skin patch.

According to still further features in the described preferred embodiments the backing comprises a plaster.

According to still further features in the described preferred embodiments the backing comprises a transparent tape.

According to still further features in the described preferred embodiments the backing comprises an adhesive tape.

According to still further features in the described preferred embodiments the medical device further comprises a removable cover for protecting the pharmaceutical composition upon storage.

According to still further features in the described preferred embodiments the medical device further comprises a protective mechanism for protecting the pharmaceutical composition against humidity upon storage.

According to still further features in the described preferred embodiments the medical device further comprises an adhesive, water permeable layer, in contact with the pharmaceutical composition.

According to still another aspect of the present invention there is provided a method of treating a skin or mucosal membranes ailment, which comprises applying onto a treated region of the skin or mucosal membranes an oxidizing agent being entrapped in or by a pharmaceutical sustained-release carrier, the carrier comprises a biocompatible polymer.

According to an additional aspect of the present invention there is provided a method of treating a skin or mucosal membranes ailment, the method comprises applying onto a treated region of the skin or mucosal membranes the medical device of the present invention.

According to yet an additional aspect of the present invention there is provided a method of treating a skin or mucosal membranes ailment, which comprises applying onto a treated region of the skin or mucosal membranes an oxidizing agent that is hydrolizable into one or more oxidizing moieties having oxidizing properties.

According to further features in preferred embodiments of the invention described below, the methods described hereinabove further comprise wetting the treated region prior to the application of the pharmaceutical composition or the medical device of the present invention.

According to still further features in the described preferred embodiments the skin ailment is caused by a microorganism.

According to still further features in the described preferred embodiments the microorganism is selected from the group consisting of a virus, bacteria and a fungi.

According to still further features in the described preferred embodiments the skin ailment is caused by a human papilloma virus.

According to still further features in the described preferred embodiments the oxidizing agent has oxidizing properties per se.

According to still further features in the described preferred embodiments the oxidizing agent is hydrolizable into one or more oxidizing moieties having oxidizing properties.

According to still further features in the described preferred embodiments the oxidizing agent comprises a chlorinated isocyanurate.

According to still further features in the described preferred embodiments the chlorinated isocyanurate is selected from the group consisting of trichloro(iso)cyanurate and sodium dichloro(iso)cyanurate.

According to still further features in the described preferred embodiments the one or more oxidizing moieties comprise free chlorine.

According to still further features in the described preferred embodiments the polymer is a conformable polymer.

According to still further features in the described preferred embodiments the polymer is a flexible polymer.

According to still further features in the described preferred embodiments the polymer is a spreadable polymer.

According to still further features in the described preferred embodiments the polymer has a form selected from the group consisting of a gel, a paste, a cream, a foam, a sheet and a solution.

According to still further features in the described preferred embodiments the polymer comprises a silicone polymer.

According to still further features in the described preferred embodiments the silicone polymer comprises a cross-linked silicone polymer.

According to still further features in the described preferred embodiments the cross-linked silicone polymer comprises a silicone rubber.

According to still further features in the described preferred embodiments the cross-linked silicone polymer is prepared by a process selected from the group consisting of a room temperature vulcanization, an elevated temperature vulcanization and a radiation.

According to still further features in the described preferred embodiments the cross-linked silicone polymer is prepared by the room temperature vulcanization of one or more silicone oil(s).

According to still further features in the described preferred embodiments the silicone polymer further comprises one or more additive(s) selected from the group consisting of a filler, a salt, a sugar, a glycerin and a glycol.

According to still further features in the described preferred embodiments the silicone polymer has a form selected from the group consisting of a gel, a paste, a cream, a foam, a sheet and a solution.

According to still further features in the described preferred embodiments the polymer or the silicone polymer is arranged in sheets.

According to still further features in the described preferred embodiments the oxidizing agent is entrapped between the sheets.

According to still further features in the described preferred embodiments the polymer or the silicone polymer is arranged in a tubular structure.

According to still further features in the described preferred embodiments the oxidizing agent is present at a concentration ranging between 10 weight % and 90 weight % of the total weight of the pharmaceutical composition.

According to still further features in the described preferred embodiments the polymer releases the oxidizing agent upon hydration, e.g., by wetting and/or body fluids, and/or diffusion.

According to a further aspect of the present invention there is provided a method of preparing a pharmaceutical composition for treating skin or mucosal membranes aliments, the method comprises polymerizing a mixture of a silicone polymer and an oxidizing agent, so as to obtain said oxidizing agent entrapped within said silicone polymer formed upon polymerization.

According to further features in preferred embodiments of the invention described below, the method further comprises polymerizing a second silicone polymer so as to obtain a second polymerized silicone polymer and filling the second polymerized silicone polymer with the mixture of the silicone polymer and the oxidizing agent.

According to yet a further aspect of the present invention there is provided a method of preparing a pharmaceutical composition for treating skin or mucosal membranes aliments, the method comprising polymerizing a silicone polymer so as to form a polymerized silicone polymer and loading the polymerized silicone polymer with an oxidizing agent, so as to obtain the oxidizing agent entrapped within the polymerized silicone polymer.

According to further features in preferred embodiments of the invention described below, the loading precedes the polymerizing.

According to still further features in the described preferred embodiments the polymerizing precedes the loading.

According to still a further aspect of the present invention there is provided a method of preparing a pharmaceutical composition for treating skin or mucosal membranes aliments, the method comprising polymerizing a silicone polymer and applying thereon an oxidizing agent.

The present invention successfully addresses the shortcomings of the presently known configurations by providing novel compositions and medical devices, which can be used in the treatment of skin and mucosal membrane ailments.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a perspective view of a silicone polymer of the present invention in a sheet form;

FIG. 2 is a perspective view of two silicone rubber sheets used to entrap the oxidizing agent therebetween, according to the present invention;

FIG. 3 is a perspective view of a sandwich configuration of a plurality of sheets of the present invention, where both the external sheets are devoid of an oxidizing agent, wherein the inner sheet contains the oxidizing agent;

FIGS. 4a and 4b are a perspective view of a silicone polymer of the present invention formed in a tubular structure (FIG. 4a) and a perspective view of circular slices derived therefrom (FIG. 4b);

FIG. 5 is a cross-sectional view of a medical device according to the present invention;

FIG. 6 is a top view of a skin patch according to the present invention; and

FIG. 7 is a top view of a plaster according to the present invention.

### DESCRIPTION OF THE PREFERRED EMBODIMENTS

The present invention is of compositions that contain a polymer, preferably, a silicone polymer, and an oxidizing agent, which can be used in the treatment of skin ailments. Specifically, the present invention is of compositions that contain a conformable and/or spreadable polymer and an oxidizing agent entrapped in or by the polymer. The compositions of the present invention can be used as pharmaceutical compositions per se or be included in a medical device. The compositions and devices of the present invention are highly effective in the treatment of skin ailments such as, but not limited to, human papilloma virus (HPV), which are best treated with conformable and/or spreadable compositions that exhibit a controlled release and a continued or repeated delivery of active ingredients.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While conceiving the present invention, it was hypothesized that oxidizing agents, which are typically known to exert an antimicrobial activity, can be used as active agents in the treatment of skin ailments that are caused by a variety of microorganisms such as viruses, bacteria or fungi.

The treatment of such skin ailments typically requires a continued topical application, which commonly lasts between a few hours and a few days, of a composition that contains a suitable agent. Therefore, it was further hypothesized that a composition that contains, in addition to an anti-microbial oxidizing agent, a conformable and/or spreadable polymer that entraps the oxidizing agent and is easily and efficiently applied onto the skin could provide for both a protection of the oxidizing agent upon storage and a sustained-release thereof upon application.

While reducing the present invention to practice, it was found that a composition containing a silicone polymer and an oxidizing agent entrapped therein or thereby, exerted high and effective activity in the treatment of skin ailments such as HPV infections. Other biocompatible polymers, which are inert with respect to the oxidizing agent employed, such as, but not limited to, hydrogels, polyisoprene, polypropylene-containing elastomers, fluoroelastomers, polyvinylchloride polymers, latex, and polyurethane or its copolymers can also be used in the context of the present invention instead of silicone.

Thus, according to one aspect of present invention there is provided a composition that comprises a polymer and an oxidizing agent entrapped in or by the polymer.

Since the composition of the present invention is aimed at treating skin and mucosal membranes ailments, polymers that are suitable for use in the context of the present invention include biocompatible polymers such as silicone polymers and other biocompatible polymers as is described herein.

The polymer of the present invention is preferably a conformable, flexible and/or spreadable polymer, and can therefore be efficiently applied onto, and form good contact with, a treated region. This feature is highly advantageous in the treatment of skin and mucosal membranes ailments since these ailments often requires continuous application of the active ingredient onto the treated region and may have varying contours.

Non-limiting examples of preferred polymers that are usable in the context of the present invention include polymers in the form of a gel, a paste, a cream, a foam, a sheet or a solution. Nevertheless, other polymers, such as rigid, elastomeric and non-elastomeric polymers are also usable in the context of the present invention.

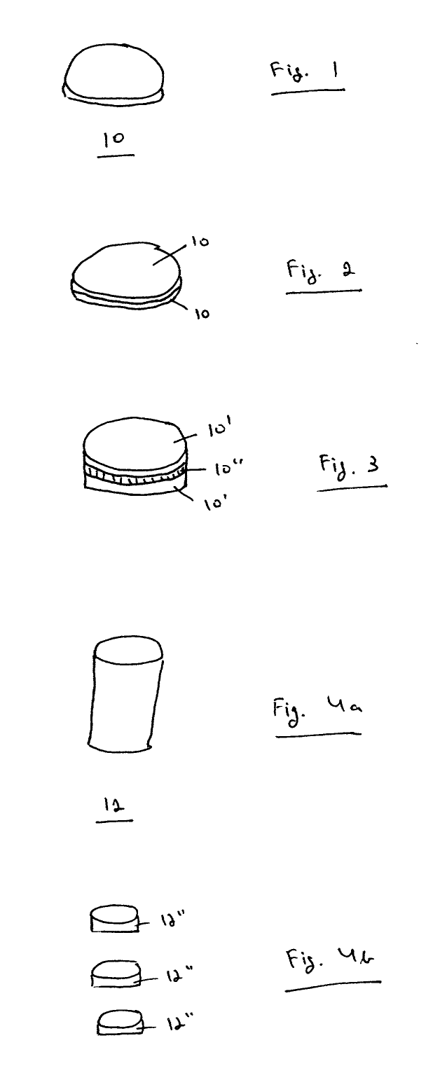

As shown in FIG. 1, preferably, the polymer of the present invention has a sheet form 10.

As shown in FIGS. 2 and 3 and is further detailed hereinbelow, further preferably, the polymer is arranged in a plurality of sheets, and the oxidizing agent is entrapped between these sheets.

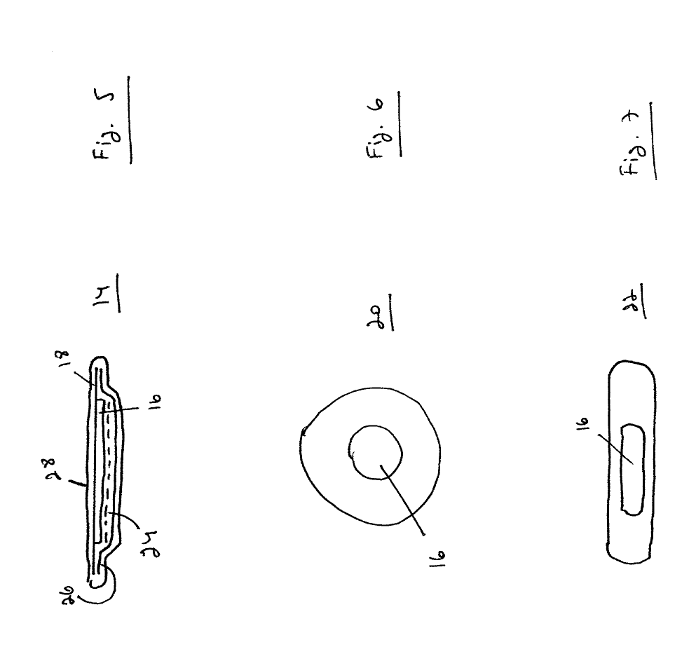

As shown in FIG. 4a, further preferably, the polymer of the present invention is acquired a tubular structure 12. As is shown in FIG. 4b and is further detailed hereinbelow, tubular structure 12 is preferably cut into circular slices 12′.

The polymeric compositions of the present invention may further include one or more additive(s), such as, but not limited to, fillers, salts, sugars, glycerin and/or glycols. These additives are typically added to the composition to reinforce the mechanical strength of the obtained polymer, to alter the diffusion or absorbency properties of the obtained polymer and/or to improve the cost-efficiency of the product.

According to a preferred embodiment of the present invention, the polymer is a silicone polymer. Silicone polymers are highly suitable polymers to be used in the compositions of the present invention, since silicone polymers are known as biocompatible materials that are permeable to gases and water vapor and are sufficiently inert so as not to interact with the active ingredients therein.

Preferred silicone polymers that are usable in the context of the present invention include, for example, silicone-based rubbers, elastomers, gels, foams and other cross-linked silicone polymers.

A cross-linked polymer is typically characterized as a thickened polymer, which is typically firmer as compared to a non-cross-linked polymer, and is therefore presently preferred in the context of the present invention.

According to a preferred embodiment of the present invention, the silicone polymer comprises a silicone rubber. Silicone rubber is a highly cross-linked polymer that is characterized, as is described hereinabove, as a flexible, elastic material that conforms to body curvature and forms a pleasant contact with the skin.

According to a preferred embodiment of the present invention, the cross-linked silicone polymer, and particularly the silicone rubber, is prepared by room temperature vulcanization (RTV).

As used herein, the phrase “room temperature vulcanization” includes polymerization of a polymeric mixture, by an addition reaction or a condensation reaction, which is performed at room temperature.

The phrase “addition reaction” refers to a polymerization reaction that involves bond formation between two functional groups. RTV addition reaction includes, for example, hydrosilylation of a vinyl silane, which is typically performed in the presence of a platinum catalyst.

The phrase “condensation reaction” refers to a polymerization reaction that involves cross-linking of functional side groups or end group, by a condensation reaction in the presence of moisture or other cross-linking agent. RTV condensation reactions are often catalyzed by organometallic compounds such as organotin compounds. Representative examples of organotin compounds include stannous octoate, di-n-octyltindilaurate and di-n-butyltindilaurate.

In an example, a silicone rubber is prepared using tin-catalyzed room temperature vulcanization of silicone oil, in the presence of a cross-linking agent such as, but not limited to, ethoxysilane.

The room temperature vulcanization technique is presently most preferable in the context of the present invention since it allows the preparation of the compositions of the present invention at room temperature, and thus allows the use of oxidizing agents that are sensitive to elevated temperatures and the use of other reaction components that are sensitive to oxidizing agents at elevated temperatures.

However, other techniques, such as elevated temperature vulcanization and radiation, can also be used to prepare the cross-linked silicone polymer of the present invention.

Non-limiting examples of elevated temperature vulcanizations include an addition reaction as described hereinabove, which is accelerated by heat and a peroxide-catalyzed reaction. The peroxide-catalyzed reaction involves thermal decomposition of organic peroxides, which generates free radicals. The generated radicals induce the formation of polymeric radicals, which form an elastomeric network upon recombination thereof.

Non-limiting examples of radiation include gamma radiation, e-beam radiation, and microwave radiation.

The polymeric mixtures that are polymerized by the techniques described hereinabove to form the cross-linked silicone polymer and, in particular, the silicone rubber, of the present invention include, for example, rubbers such as, but not limited to, high consistency rubbers, low consistency rubbers and liquid rubbers, dispersions or silicone oil mixtures.

The polymeric mixtures may further include one or more filler(s) such as, but not limited to, a silica filler, a quartz filler, a diatomaceous earth or calcium carbonate. The polymeric mixtures may further include other additives, as described hereinabove.

The final form of the silicone polymer of the present invention may be, for example, a gel, a foam, a paste, a cream, a sheet or a solution.

The polymers of the present invention may further include interstitial spaces or pores and therefore allow the entrapment of an oxidizing agent within the spaces or pores. The polymers can be further prepared having a rough surface characterized by surface formed grooves, which can hold the oxidizing agent therein. Loading a rough surface characterized by surface formed grooves can be effected post manufacture of the rough surface.

As used herein, the phrase “oxidizing agent” includes a compound or a mixture of compounds that exerts oxidizing properties either directly or indirectly.

Thus, the oxidizing agent, according to the present invention, can be an oxidizing agent that has oxidizing properties per se or, optionally and preferably, an oxidizing agent that is hydrolizable into at least one oxidizing moiety that has oxidizing properties.

An oxidizing agent that is capable of producing one or more oxidizing moieties upon hydrolysis is presently preferred over an oxidizing agent that has oxidizing properties per se since oxidizing agents are typically highly active, aggressive and non-stable reagents and therefore the preparation, handling and use of compositions containing same is limited. The use of a hydrolizable oxidizing agent as described above, provides for a release of the active oxidizing moiety(ies) only upon hydration and thus provides for more convenient preparation and handling prior to its use, and further for safer use.

Hence, the hydrolizable oxidizing agent, according to the present invention, is preferably selected so as to generate one or more oxidizing moieties upon hydration by water, water vapor or other fluids present in a treated region.

Oxidizing agents that have oxidizing properties per se, which are suitable for use in context of the present invention include, for example, agents that exert anti-microbial, anti-viral and anti-fungal activities such as, but not limited to, hypochlorous acid and other hypohalous acids, elemental iodine and hydrogen peroxide.

Oxidizing agents that are hydrolizable into one or more oxidizing moieties having oxidizing properties, which are suitable for use in context of the present invention include, for example, reagents that are capable of producing the oxidizing moieties described hereinabove upon hydration.

According to a preferred embodiment of the present invention, the oxidizing agent is a chlorinated isocyanurate. Chlorinated isocyanurates, such as the commercially available trichloro(iso)cyanurate (TCIA) and sodium dichloro(iso)cyanurate (DCIA), are known to react with water and produce hypochlorous acid, as shown, for example, in Scheme I below.

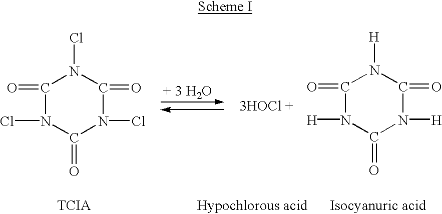

Hypochlorous acid is a common source of “free chlorine”, a phrase which is used herein to describe the active halonium compound [Cl⁺], and is therefore well known in the art as an aggressive oxidizing and chlorinating agent. Presently, hypochlorous acids, as well as its parent compounds, e.g., TCIA and DCIA, are used in water purification systems, since the “free chlorine” generated by the hypochlorous acid is well known as an effective anti-microbial, anti-fungal and anti-viral agent.

Thus, a composition that comprises a chlorinated cyanurate, as described hereinabove, that is hydrolizable, upon hydration, into a hypochlorous acid, which is an efficient anti-microbial, anti-fungal and anti-viral agent, can serve as an efficient agent in the treatment of skin ailment caused by a microorganism such as virus, bacteria and/or fungi.

The oxidizing agent is present in the composition of the present invention at a concentration ranging between 10 weight % and 90 weight %, preferably between 20 weight % and 80 weight %, most preferably between 40 weight % and 60 weight % of the total weight of the composition.

The oxidizing agent, according to the present invention, is either entrapped in or by a polymer.

As used herein, the phrase “an oxidizing agent entrapped in or by a polymer” refers to an oxidizing agent that is entrapped within the polymeric matrix of the polymer and/or an oxidizing agent that is spread over a surface or between surfaces of polymer sheets.

Thus, in one alternative, the composition of the present invention comprises a silicone rubber that is molded and cured as a sheet (10, FIG. 1) or as a tubular structure (12, FIG. 4). The oxidizing agent is mixed with the silicone polymer prior to its molding and curing and is therefore entrapped within the polymeric matrix formed upon curing.

In another alternative, the oxidizing agent is added onto the silicone rubber sheet or tubular structure after its molding and curing and is therefore entrapped within interstitial spaces or grooves formed in the surface of the sheet. It will be appreciated that both alternatives may be practiced for the same composition. In addition, and optionally, as shown in FIG. 2, two or more silicone rubber sheets 10 are used to entrap the oxidizing agent therebetween. As shown in FIG. 3, various sandwich configurations of a plurality of sheets may be employed, wherein one or both external sheets 10′ of a sandwich configuration are devoid of the oxidizing agent, wherein inner sheets 10″ contain the oxidizing agent.

In yet another alternative, the composition of the present invention comprises an oxidizing agent mixed with a silicone rubber. The composition is loaded into another silicone composition, such as a silicone rubber, that is molded and cured as a tubular structure or as a sheet having a depression. The composition is further cured thereafter, and the oxidizing agent is therefore entrapped within the polymeric matrix formed upon curing.

Compositions that are molded and cured as a tubular structure, as described hereinabove, can be later cut into circular slices (12′, FIG. 4b). The cut slices are characterized by high content of the entrapped oxidizing agent on their surfaces and are therefore highly advantageous in the context of the present invention.

Hence, according to another aspect of the present invention, there is provided a pharmaceutical composition that comprises, as an active ingredient, an oxidizing agent as defined hereinabove, which is entrapped in or by a pharmaceutical sustained-release carrier that comprises any of the polymers described hereinabove.

Thus, the pharmaceutical composition according to the present invention includes a polymer which serves as a carrier which provides a sustained release effect of the active oxidizing agent.

According to a preferred embodiment of the present invention, the polymer releases the active oxidizing agent upon hydration and/or diffusion. The hydration of the active oxidizing agent includes hydrolysis of the oxidizing agent entrapped in or by the polymer into the oxidizing moiety(ies), which is followed by diffusion of the oxidizing moiety(ies) from the composition. The diffusion includes a release of the active oxidizing agent from the composition as a result of a concentration gradient, which can be followed by its hydrolysis into the active oxidizing moiety(ies).

In one example, the pharmaceutical composition of the present invention comprises a TCIA entrapped in or by a silicone rubber carrier. Upon hydration, e.g., by contacting water, water vapor or other fluids present in a treated region (e.g., sweat) following placement on the treated region, the TCIA is hydrolyzed into hypochlorous acid. Since the hypochlorous acid molecules are small, they diffuse out of the carrier.

Alternatively, the entrapped TCIA diffuses from the carrier upon placement on a treated region, due to a concentration gradient (e.g., high level of TCIA in the composition and low level of TCIA in the treated region). Upon contacting water, water vapor or other fluids present in the treated region, the TCIA is hydrolyzed into hypochlorous acid.

Thus, by pre-determining the properties of the carrier (e.g., the cross-linking degree of a silicone rubber carrier or the presence and size of pores within the carrier) and the amount of the oxidizing agent entrapped in or by the carrier, the rate of diffusion and the sustained release of the oxidizing agent can be controlled as desired.

The pharmaceutical composition of the present invention is highly effective in the treatment of skin ailments, in particular skin ailments that are caused by microorganisms such as, but not limited to, bacteria, viruses or fungi, all are highly sensitive to the oxidizing agents employed by the present invention. In many cases, the treatment of skin ailments requires a repeated or continuous topical application, e.g., of at least several hours, of the active agent. Hence, a pharmaceutical composition that assures sustained release of an oxidizing agent upon contacting the skin is advantageous.

According to a preferred embodiment of the present invention, the pharmaceutical composition is packaged and identified for the treatment of skin or mucosal membranes ailments, in particular for a treatment of the skin ailments described hereinabove and, further in particular, for a treatment of HPV infections.

As is further detailed in the Examples section that follows, a pharmaceutical composition of the present invention is highly effective in the treatment of verruca, which is a common type of HPV infection. Verrucas that were treated with the pharmaceutical composition of the present invention, as is further described hereinafter, completely disappeared and did not reappear after a long period of time (e.g., several months). These results emphasize the effectiveness of the compositions of the present invention and their advantage over the presently used compositions, which are often either unsuccessful or require repeated treatments.

Thus, according to another aspect of the present invention, there is provided a method of treating a skin or mucosal membranes ailment. The method is effected by applying onto a treated region of the skin or mucosal membranes the pharmaceutical composition of the present invention as described hereinabove.

According to a preferred embodiment of the present invention, the method further comprises wetting the treated region prior to applying the composition thereon. The step of wetting is aimed either at encouraging the release of the active oxidizing agent or moiety(ies) from the composition, as described hereinabove and thus delivering the oxidizing agent or moiety(ies) to the treated region, and/or at hydrolyzing the oxidizing agent upon its diffusion from the composition, thus producing the active oxidizing moiety(ies). Such a release can be also effected without pre-wetting the treated skin or composition, as sweat or other fluids in the treated region, such as fluids present in the body of a skin wart, will tend to accumulate and wet the composition or the oxidizing agent of the invention.

As shown in FIG. 5, according to another aspect of the present invention, there is provided a medical device 14 that comprises the pharmaceutical composition 16 of the present invention, as is described hereinabove. Preferably, the medical device has a flat configuration. A flat configuration provides for high surface area of the medical device and therefore provides for improved contact between the medical device and an affected region that undergoes treatment. Furthermore, a flat configuration provides for faster diffusion of the oxidizing agent to the surface of the medical device and thus ensures efficient application onto the treated region.

According to a preferred embodiment of the present invention, the medical device comprises a backing 18. The backing is aimed at backing the pharmaceutical composition when applied onto the treated region, so as to provide improved and stable contact between the composition and the treated region and further to protect the composition from external factors when stored or applied. The backing may also serve to provide the device with sufficient structural rigidity. The backing employed can be a transparent tape, which is highly advantageous for medical devices designed for self-use. As shown in FIGS. 6 and 7, the medical device of the present invention may be designed as a skin patch 20 or a plaster 22 form, to which composition 16 is attached.

As is shown in FIG. 5, the device of the present invention can further include a frontal water or water vapor permeable adhesive 24, which adheres and secures the device to the treated region. Silicone rubbers with self-adhesiveness can alternatively be used. In both cases, a protective permeable layer 26 can be used to protect the adhesive side of the device prior to its use. As is further shown in FIG. 5, according to another preferred embodiment of the present invention, the medical device further comprises a removable cover 28, which serves for protecting the pharmaceutical composition upon storage. The removable cover can be of any inert material unreactive with the oxidizing agent. It is also preferably water impermeable. Various plastics are applicable.

Thus, the medical device of the present invention preferably comprises a protective mechanism to protect the composition therein against humidity upon storage.

An example of a medical device according to the present invention includes a pharmaceutical composition that comprises an oxidizing agent such as TCIA entrapped in or by a silicone rubber and prepared as a cut sheet, which is placed on a pressure sensitive backing of a transparent tape.

Another example of a medical device according to the present invention includes a pharmaceutical composition that comprises an oxidizing agent such as TCIA entrapped in or by a silicone rubber and prepared as a slice of a preformed tubular structure, which is placed on a pressure sensitive backing of a transparent tape. Optionally, a medical device according to the present invention includes an extruded silicone tubular structure, which is filled by the pharmaceutical composition described hereinabove and thereafter sliced and placed on a pressure sensitive backing of a transparent tape. As is discussed hereinabove, a medical device that includes a pharmaceutical composition as described hereinabove, which is prepared as a sliced tubular structure, is preferable.

The medical device of the present invention can be used to treat skin and mucosal membrane ailments such as, but not limited to, the ailments described herein, by placement over an inflicted skin region.

As so far described, the present invention provides novel compositions that comprise a polymer and an oxidizing agent entrapped in or by the polymer, novel medical devices containing these compositions and methods of treating skin or mucosal membranes ailments using the compositions per se or the medical devices including same.

However, since the active ingredient of the pharmaceutical compositions of the present invention is the oxidizing agent, there is provided, according to another aspect of the present invention, another method of treating such ailments, which is effected by applying onto a treated region of the skin or mucosal membranes an oxidizing agent which is hydrolizable into one or more oxidizing moieties having oxidizing properties, as these terms are defined hereinabove. Preferably, the method is further effected by wetting the treated region prior to applying the oxidizing agent thereon, so as to hydrolyze the oxidizing agent into the active oxidizing moieties. The oxidizing agent, according to this method of the present invention, can be in a solid form, e.g., a powder, and is thus spread over the treated region. Optionally, the oxidizing agent can be in a liquid form and can be applied onto the treated region by means of a dipped cotton wool, for example.

The present invention further provides methods of preparing a pharmaceutical composition for treating skin or mucosal membranes aliments.

In one method, the pharmaceutical composition is prepared by polymerizing a mixture of a silicone polymer and an oxidizing agent, so as to obtain the oxidizing agent entrapped within the silicone polymer formed upon polymerization. This method can further comprise polymerizing a second, oxidizing agent-free silicone polymer and filling the silicone polymer formed upon this polymerization with the mixture of the silicone polymer and the oxidizing agent.

Alternatively, the pharmaceutical composition is prepared by the polymerizing a silicone polymer so as to form a polymerized silicone polymer and thereafter loading the polymerized silicone polymer with an oxidizing agent, so as to entrap the agent within the polymerized silicone polymer.

In another method, the pharmaceutical composition is prepared by polymerizing a silicone polymer and applying thereon an oxidizing agent.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### MATERIALS AND METHODS

Preparation of compositions containing silicone polymer and an oxidizing agent—general procedures: Representative examples of the compositions of the present invention include tin-catalyzed room-temperature-vulcanizing (RTV) biocompatible silicones and an oxidizing agent which comprises 20-80 weight % of the final composition.

Compositions containing an oxidizing agent entrapped in a silicone polymer: Hydroxy-terminated, linear polydimethylsiloxane (PDMS) having a molecular weight of about 5000 mPa·s, which is also known as silicone oil, and an amount of an oxidizing agent calculated according to its desired final concentration in the composition, are stirred for about 30-60 seconds. About 0.5% (weight % of the silicone oil) of a cross-linking agent, such as ethoxysilane, is then added, and about 1% (weight % of the silicone oil) of a tin catalyst, such as di-n-octyltindilaurate catalyst, and a minute amount of distilled water are thereafter added dropwise and the mixture is stirred again for about 30 seconds. The mixture is then poured or spread in an open mold having a shape and thickness as desired. The obtained composition cures at room temperature within a few hours and can be optionally allowed to fully cure for up to 24 hours.

Alternatively, an oxidizing agent-free silicone composition, such as a silicone rubber, having a shape and thickness as desired, is formed, by means such as molding or extrusion, and cured. The obtained composition is then loaded with an oxidizing agent or with a composition containing an oxidizing agent entrapped in a silicone polymer, prepared as described hereinabove, by means of spreading, coating, dropping or filling.

Composition containing an oxidizing agent entrapped by a silicone polymer: An oxidizing agent or a composition containing same, prepared as described hereinabove, is encapsulated between two sheets of oxidizing agent-free silicone rubber, prepared by room temperature vulcanization.

Composition containing an oxidizing agent entrapped in a plurality of silicone polymer sheets: A silicone sheet having a thickness of about 1 mm and containing as oxidizing agent entrapped therein, prepared as described hereinabove, is pressed between two layers of active-agent free silicone rubber.

Preparation of a composition containing trichloro(iso)cyanurate (TCIA) entrapped in a silicone polymer (TCIA composition): Hundred grams of silicone oil, as described hereinabove, and the desired amount of TCIA, were stirred for about 30-60 seconds. Exemplary amounts of TCIA that were used in the compositions of the present invention include 26 grams, 68 grams and 150 grams, which served to obtain final TCIA weight fractions of 20%, 40% and 60%, respectively. One gram ethoxysilane cross-linking agent, 0.2 gram di-n-octyltindilaurate catalyst and 0.1 gram distilled water were thereafter added dropwise and the mixture was stirred for additional 30 seconds. The obtained mixture was then poured or spread in an open mold having a shape and thickness as desired. The obtained composition cured at room temperature within a few hours and was allowed to fully cure for up to 24 hours.

Preparation of a medical device containing a TCIA composition: Circles of a TCIA composition, prepared as described hereinabove, which have a 5 mm diameter and 1 mm thickness, were obtained by molding, were cut from larger molded sheets, were sliced from molded tubular structures or were sliced from silicone extruded tubular structures filled with a TCIA composition. The circles were placed and centered on a pressure sensitive backing of a transparent tape, which was cut to circular shapes having about 20 mm diameter.

Preparation of a medical device containing a composition of sodium dichloro(iso)cyanurate entrapped in a plurality of silicone rubber sheets: A silicone sheet having a thickness of about 1 mm and containing 80% DCIA, prepared according to the general procedure described hereinabove, was pressed between two 0.2 mm layers of active-agent free silicone rubber. The obtained triple-layered sheet was cut into patches having a diameter of about 8 mm. The patches are attachable to a wart, upon wetting thereof, by means of a pressure sensitive adhesive tape.

Treatment of skin warts using TCIA compositions or compositions containing DCIA and a silicone polymer—general method: A silicone composition or a medical device, prepared according to any of the procedures described hereinabove, were applied for several hours onto a skin growth that typically resulted from verruca, preferably after wetting the affected skin area. The treated area of the skin was re-examined regularly every few months.

### EXPERIMENTAL RESULTS

A skin growth having a diameter of about 2.5 mm and a height of about 1.5 mm, present for about 2 years on the hand of a 50 years old woman, was treated with a TCIA composition of the present invention. The skin growth disappeared completely after one treatment. After 9 months, the warts did not reappear in the treated skin area.

An area of about 4 mm×2 mm of a TCIA composition of the present invention, which contained 60% weight TCIA, was cut. A skin wart having a diameter of about 2 mm and a height of about 1-1.5 mm, present for a few weeks, was opened slightly with tweezers, wetted the TCIA composition was applied thereon. The composition was then secured against the wart growth by means of a transparent adhesive tape. After about 6 hours of exposure to the composition on one day and an additional 3-4 hours of exposure on the following day, the growth root was revealed. After additional exposure of 3-4 hours, the growth disappeared completely. The TCIA composition was then removed. After about 1-2 weeks no traces of the wart were detected. After 9 months, the warts did not reappear in the treated area.

A 40 years old woman that had three different growths on her hand, for about one year, was treated. The growth on her index finger had an initial diameter of about 2.5 mm and the growths on her ring finger and small finger had a diameter of about 2 mm and 1.5 mm, respectively. Prior to the treatment, the woman routinely cut the growths flat with scissors. Each of the growths was slightly wetted with water and a 60% TCIA composition of the present invention and secured with a transparent adhesive tape. After 18 hours of exposure to this treatment, the entire growth on the small finger turned blood red, while the root of the growth on the ring finger turned black. Within one week, and after an additional treatment of about 3-4 hours, the small finger growth disappeared completely while the ring finger black root became slightly smaller and white and the overall growth was slightly smaller than its original size. The index finger growth was not effected by these treatments. After about 1-2 weeks, the TCIA composition was removed. No trace of the small finger wart was detected thereafter. After 9 months, no regrowth of the warts was apparent in the treated area.

A 40 years old man had a growth of about 4 mm diameter. The affected skin area was treated with a composition as above. During the first 3 days, the affected skin area was treated one or two times for about 5-6 hours. As a result, the size of the growth was reduced to approximately half of its original size. During the next two weeks, the growth was treated another one or two times and, as a result, the growth size was reduced to about 1 mm diameter. After 9 month, no regrowth of the warts was apparent in the treated area. A patch having a thickness of 1 mm and a diameter of 3 mm, which contained 40% TCIA composition of the present invention, was applied on a wart of an 8 years old girl during two periods of 1.5-2 hours each. After removing the patch, the wart came out of the skin completely. No trace of the wart remained after a period of about 1-2 weeks. After 9 months, no regrowth of the warts was apparent in the treated area.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A composition-of-matter comprising a polymer and an oxidizing agent being entrapped in or by said polymer.

2. The composition-of-matter of claim 1, wherein said polymer is a conformable polymer.

3. The composition-of-matter of claim 1, wherein said polymer is a flexible polymer.

4. The composition-of-matter of claim 1, wherein said polymer is a spreadable polymer.

5. The composition-of-matter of claim 1, wherein said polymer has a form selected from the group consisting of a gel, a paste, a cream, a foam, a sheet and a solution.

6. The composition-of-matter of claim 1, wherein said polymer is arranged in at least one sheet.

7. The composition-of-matter of claim 1, wherein said polymer is arranged in a plurality of sheets, whereas said oxidizing agent is entrapped between said sheets.

8. The composition-of-matter of claim 1, wherein said polymer is arranged in a tubular structure.

9. The composition-of-matter of claim 1, further comprising at least one additive selected from the group consisting of a filler, a salt, a sugar, a glycerin and a glycol.

10. The composition-of-matter of claim 1, wherein said oxidizing agent has oxidizing properties per se.

11. The composition-of-matter of claim 1, wherein said oxidizing agent is hydrolizable into at least one oxidizing moiety having oxidizing properties.

12. The composition-of-matter of claim 11, wherein said oxidizing agent comprises a chlorinated isocyanurate.

13. The composition-of-matter of claim 12, wherein said chlorinated isocyanurate is selected from the group consisting of trichloro(iso)cyanurate and sodium dichloro(iso)cyanurate.

14. The composition-of-matter of claim 12, wherein said at least one oxidizing moiety comprises free chlorine.

15. The composition-of-matter of claim 1, wherein said polymer is a silicone polymer.

16. The composition-of-matter of claim 15, wherein said silicone polymer comprises a cross-linked silicone polymer.

17. The composition-of-matter of claim 16, wherein said cross-linked silicone polymer comprises a silicone rubber.

18. The composition-of-matter of claim 16, wherein said cross-linked silicone polymer is prepared by a process selected from the group consisting of a room temperature vulcanization, an elevated temperature vulcanization and a radiation.

19. The composition-of-matter of claim 18, wherein said cross-linked silicone polymer is prepared by said room temperature vulcanization of at least one silicone oil.

20. The composition-of-matter of claim 15, wherein said silicone polymer further comprises at least one additive selected from the group consisting of a filler, a salt, a sugar, a glycerin and a glycol.

21. The composition-of-matter of claim 15, wherein said silicone polymer has a form selected from the group consisting of a gel, a paste, a cream, a foam, a sheet and a solution.

22. The composition-of-matter of claim 15, wherein said silicone polymer is arranged in at least one sheet.

23. The composition-of-matter of claim 15, wherein said silicone polymer is arranged in a plurality of sheets, whereas said oxidizing agent is entrapped between said sheets.

24. The composition-of-matter of claim 15, wherein said silicone polymer is arranged in a tubular structure.

25. The composition-of-matter of claim 1, wherein said oxidizing agent is present at a concentration ranging between 10 weight % and 90 weight % of the total weight of said composition.

26. A pharmaceutical composition comprising, as an active ingredient, an oxidizing agent being entrapped in or by a pharmaceutical sustained-release carrier, said carrier comprises a polymer.

27. The pharmaceutical composition of claim 26, wherein said polymer is a conformable polymer.

28. The pharmaceutical composition of claim 26, wherein said polymer is a flexible polymer.

29. The pharmaceutical composition of claim 26, wherein said polymer is a spreadable polymer.

30. The pharmaceutical composition of claim 26, wherein said polymer has a form selected from the group consisting of a gel, a paste, a cream, a foam, a sheet and a solution.

31. The pharmaceutical composition of claim 26, wherein said polymer is arranged in at least one sheet.

32. The pharmaceutical composition of claim 26, wherein said polymer is arranged in a plurality of sheets, whereas said oxidizing agent is entrapped between said sheets.

33. The pharmaceutical composition of claim 26, wherein said polymer is arranged in a tubular structure.

34. The pharmaceutical composition of claim 26, further comprising at least one additive selected from the group consisting of a filler, a salt, a sugar, a glycerin and a glycol.

35. The pharmaceutical composition of claim 26, packaged and identified for the treatment of a skin or mucosal membranes ailment.

36. The pharmaceutical composition of claim 35, wherein said skin ailment is caused by a microorganism.

37. The pharmaceutical composition of claim 36, wherein said microorganism is selected from the group consisting of a virus, bacteria and a fungi.

38. The pharmaceutical composition of claim 35, wherein said skin ailment is caused by a human papilloma virus.

39. The pharmaceutical composition of claim 26, wherein said oxidizing agent has oxidizing properties per se.

40. The pharmaceutical composition of claim 26, wherein said oxidizing agent is hydrolizable into at least one oxidizing moiety having oxidizing properties.

41. The pharmaceutical composition of claim 40, wherein said oxidizing agent comprises a chlorinated isocyanurate.

42. The pharmaceutical composition of claim 41, wherein said chlorinated isocyanurate is selected from the group consisting of trichloro(iso)cyanurate and sodium dichloro(iso)cyanurate.

43. The pharmaceutical composition of claim 41, wherein said at least one oxidizing moiety comprises free chlorine.

44. The pharmaceutical composition of claim 26, wherein said polymer is a silicone polymer.

45. The pharmaceutical composition of claim 44, wherein said silicone polymer comprises a cross-linked silicone polymer.

46. The pharmaceutical composition of claim 44, wherein said cross-linked silicone polymer comprises a silicone rubber.

47. The pharmaceutical composition of claim 45, wherein said cross-linked silicone polymer is prepared by a process selected from the group consisting of a room temperature vulcanization, an elevated temperature vulcanization and a radiation.

48. The pharmaceutical composition of claim 47, wherein said cross-linked silicone polymer is prepared by said room temperature vulcanization of at least one silicone oil.

49. The pharmaceutical composition of claim 45, wherein said silicone polymer further comprises at least one additive selected from the group consisting of a filler, a salt, a sugar, a glycerin and a glycol.

50. The pharmaceutical composition of claim 45, wherein said silicone polymer has a form selected from the group consisting of a gel, a paste, a cream, a foam, a sheet and a solution.

51. The pharmaceutical composition of claim 44, wherein said silicone polymer is arranged in at least one sheet.

52. The pharmaceutical composition of claim 44, wherein said silicone polymer is arranged in a plurality of sheets, whereas said oxidizing agent is entrapped between said sheets.

53. The pharmaceutical composition of claim 44, wherein said silicone polymer is arranged in a tubular structure.

54. The pharmaceutical composition of claim 26, wherein said oxidizing agent is present at a concentration ranging between 10 weight % and 90 weight % of the total weight of said pharmaceutical composition.

55. The pharmaceutical composition of claim 26, wherein said polymer releases said oxidizing agent upon hydration and/or diffusion.

56. The pharmaceutical composition of claim 55, wherein said hydration is effectable by body fluids.

57. A method of treating a skin or mucosal membranes ailment, the method comprising applying onto a treated region of the skin or mucosal membranes an oxidizing agent being entrapped in or by a pharmaceutical sustained-release carrier, said carrier comprises a biocompatible polymer.

58. The method of claim 57, wherein said biocompatible polymer is a conformable polymer.

59. The method of claim 57, wherein said biocompatible polymer is a flexible polymer.

60. The method of claim 57, wherein said biocompatible polymer is a spreadable polymer.

61. The method of claim 57, wherein said biocompatible polymer has a form selected from the group consisting of a gel, a paste, a cream, a foam, a sheet and a solution.

62. The method of claim 57, wherein said biocompatible polymer is arranged in at least one sheet.

63. The method of claim 57, wherein said biocompatible polymer is arranged in a plurality of sheets, whereas said oxidizing agent is entrapped between said sheets.

64. The method of claim 57, wherein said biocompatible polymer is arranged in a tubular structure.

65. The method of claim 57, further comprising wetting said treated region prior to said applying.

66. The method of claim 57, wherein said skin ailment is caused by a microorganism.

67. The method of claim 66, wherein said microorganism is selected from the group consisting of a virus, bacteria and a fungi.

68. The method of claim 57, wherein said skin ailment is caused by a human papilloma virus.

69. The method of claim 57, wherein said oxidizing agent has oxidizing properties per se.

70. The method of claim 57, wherein said oxidizing agent is hydrolizable into at least one oxidizing moiety having oxidizing properties.

71. The method of claim 70, wherein said oxidizing agent comprises a chlorinated isocyanurate.

72. The method of claim 71, wherein said chlorinated isocyanurate is selected from the group consisting of trichloro(iso)cyanurate and sodium dichloro(iso)cyanurate.

73. The method of claim 71, wherein said at least one oxidizing moiety comprises free chlorine.

74. The method of claim 57, wherein said biocompatible polymer comprises a silicone polymer.

75. The method of claim 74, wherein said silicone polymer comprises a cross-linked silicone polymer.

76. The method of claim 74, wherein said cross-linked silicone polymer comprises a silicone rubber.

77. The method of claim 75, wherein said cross-linked silicone polymer is prepared by a process selected from the group consisting of a room temperature vulcanization, an elevated temperature vulcanization and a radiation.

78. The method of claim 77, wherein said cross-linked silicone polymer is prepared by said room temperature vulcanization of at least one silicone oil.

79. The method of claim 74, wherein said silicone polymer further comprises at least one additive selected from the group consisting of a filler, a salt, a sugar, a glycerin and a glycol.

80. The method of claim 74, wherein said silicone polymer has a form selected from the group consisting of a gel, a paste, a cream, a foam, a sheet and a solution.

81. The method of claim 74, wherein said silicone polymer is arranged in at least one sheet.

82. The method of claim 74, wherein said silicone polymer is arranged in a plurality of sheets, whereas said oxidizing agent is entrapped between said sheets.

83. The method of claim 74, wherein said silicone polymer is arranged in a tubular structure.

84. The method of claim 57, wherein said biocompatible polymer releases said oxidizing agent upon hydration and/or diffusion.

85. The method of claim 84, wherein said hydration is effectable by body fluids.

86. A medical device being designed and shaped to be applied onto a skin of a subject in need, comprising a pharmaceutical composition, which comprises, as an active ingredient, an oxidizing agent being entrapped in or by a pharmaceutical sustained-release carrier, said carrier comprises a biocompatible polymer.

87. The medical device of claim 86, wherein said biocompatible polymer is a conformable polymer.

88. The medical device of claim 86, wherein said biocompatible polymer is a flexible polymer.

89. The medical device of claim 86, wherein said biocompatible polymer is a spreadable polymer.

90. The medical device of claim 86, wherein said biocompatible polymer has a form selected from the group consisting of a gel, a paste, a cream, a foam, a sheet and a solution.

91. The medical device of claim 86, wherein said biocompatible polymer is arranged in at least one sheet.

92. The medical device of claim 86, wherein said biocompatible polymer is arranged in a plurality of sheets, whereas said oxidizing agent is entrapped between said sheets.

93. The medical device of claim 86, wherein said biocompatible polymer is arranged in a tubular structure.

94. The medical device of claim 86, having a flat configuration.

95. The medical device of claim 86, further comprising a backing for backing said pharmaceutical composition when applied.

96. The medical device of claim 95, wherein said medical device is a skin patch.

97. The medical device of claim 95, wherein said backing comprises a plaster.

98. The medical device of claim 95, wherein said backing comprises a transparent tape.

99. The medical device of claim 95, wherein said backing comprises an adhesive tape.

100. The medical device of claim 86, further comprising a removable cover for protecting said pharmaceutical composition upon storage.

101. The medical device of claim 86, further comprising a protective mechanism for protecting said pharmaceutical composition against humidity upon storage.

102. The medical device of claim 86, further comprising an adhesive, water permeable layer, in contact with said pharmaceutical composition.

103. The medical device of claim 86, wherein said oxidizing agent has oxidizing properties per se.

104. The medical device of claim 86, wherein said oxidizing agent is hydrolizable into at least one oxidizing moiety having oxidizing properties.

105. The medical device of claim 104, wherein said oxidizing agent comprises a chlorinated isocyanurate.

106. The medical device of claim 105, wherein said chlorinated isocyanurate is selected from the group consisting of trichloro(iso)cyanurate and sodium dichloro(iso)cyanurate.

107. The medical device of claim 105, wherein said at least one oxidizing moiety comprises free chlorine.

108. The medical device of claim 86, wherein said biocompatible polymer comprises a silicone polymer.

109. The medical device of claim 108, wherein said silicone polymer comprises a cross-linked silicone polymer.

110. The medical device of claim 108, wherein said cross-linked silicone polymer comprises a silicone rubber.

111. The medical device of claim 109, wherein said cross-linked silicone polymer is prepared by a process selected from the group consisting of a room temperature vulcanization, an elevated temperature vulcanization and a radiation.

112. The medical device of claim 111, wherein said cross-linked silicone polymer is prepared by said room temperature vulcanization of at least one silicone oil.

113. The medical device of claim 108, wherein said silicone polymer further comprises at least one additive selected from the group consisting of a filler, a salt, a sugar, a glycerin and a glycol.

114. The medical device of claim 108, wherein said silicone polymer has a form selected from the group consisting of a gel, a paste, a cream, a foam, a sheet and a solution.

115. The medical device of claim 108, wherein said silicone polymer is arranged in at least one sheet.

116. The medical device of claim 108, wherein said silicone polymer is arranged in a plurality of sheets, whereas said oxidizing agent is entrapped between said sheets.

117. The medical device of claim 108, wherein said silicone polymer is arranged in a tubular structure.

118. The medical device of claim 86, wherein said oxidizing agent is present at a concentration ranging between 10 weight % and 90 weight % of the total weight of said pharmaceutical composition.

119. The medical device of claim 86, wherein said biocompatible polymer releases said oxidizing agent upon hydration and/or diffusion.

120. The medical device of claim 119, wherein said hydration is effectable by body fluids.

121. A method of treating a skin or mucosal membranes ailment, the method comprising applying onto a treated region of the skin or mucosal membranes a medical device that comprises a pharmaceutical composition, which comprises, as an active ingredient, an oxidizing agent being entrapped in or by a pharmaceutical sustained-release carrier, said carrier comprises a biocompatible polymer.

122. The method of claim 121, wherein said biocompatible polymer is a conformable polymer.

123. The method of claim 121, wherein said biocompatible polymer is a flexible polymer.

124. The method of claim 121, wherein said biocompatible polymer is a spreadable polymer.

125. The method of claim 121, wherein said polymer has a form selected from the group consisting of a gel, a paste, a cream, a foam, a sheet and a solution.

126. The method of claim 121, wherein said biocompatible polymer is arranged in at least one sheet.

127. The method of claim 121, wherein said biocompatible polymer is arranged in a plurality of sheets, whereas said oxidizing agent is entrapped between said sheets.

128. The method of claim 121, wherein said biocompatible polymer is arranged in a tubular structure.

129. The method of claim 121, wherein said skin ailment is caused by a microorganism.

130. The method of claim 129, wherein said microorganism is selected from the group consisting of a virus, bacteria and a fungi.

131. The method of claim 121, wherein said skin ailment is caused by a human papilloma virus.

132. The method of claim 121, further comprising wetting said treated region prior to said applying.

133. The method of claim 121, wherein said oxidizing agent has oxidizing properties per se.

134. The method of claim 121, wherein said oxidizing agent is hydrolizable into at least one oxidizing moiety having oxidizing properties.

135. The method of claim 134, wherein said oxidizing agent comprises a chlorinated isocyanurate.

136. The method of claim 135, wherein said chlorinated isocyanurate is selected from the group consisting of trichloro(iso)cyanurate and sodium dichloro(iso)cyanurate.

137. The method of claim 135, wherein said at least one oxidizing moiety comprises free chlorine.

138. The method of claim 121, wherein said biocompatible polymer comprises a silicone polymer.

139. The method of claim 138, wherein said silicone polymer comprises a cross-linked silicone polymer.

140. The method of claim 138, wherein said cross-linked silicone polymer comprises a silicone rubber.

141. The method of claim 139, wherein said cross-linked silicone polymer is prepared by a process selected from the group consisting of a room temperature vulcanization, an elevated temperature vulcanization and a radiation.

142. The method of claim 141, wherein said cross-linked silicone polymer is prepared by said room temperature vulcanization of a silicone oil.

143. The method of claim 138, wherein said silicone polymer further comprises at least one additive selected from the group consisting of a filler, a salt, a sugar, a glycerin and a glycol.

144. The method of claim 138, wherein said silicone polymer has a form selected from the group consisting of a gel, a paste, a cream, a foam, a sheet and a solution.

145. The method of claim 138, wherein said silicone polymer is arranged in at least one sheet.

146. The method of claim 138, wherein said silicone polymer is arranged in a plurality of sheets, whereas said oxidizing agent is entrapped between said sheets.

147. The method of claim 138, wherein said silicone polymer is arranged in a tubular structure.

148. The method of claim 121, wherein said biocompatible polymer releases said oxidizing agent upon hydration and/or diffusion.

149. The method of claim 148, wherein said hydration is effectable by body fluids.

150. A method of treating a skin or mucosal membranes ailment, the method comprising applying onto a treated region of the skin or mucosal membranes an oxidizing agent, said oxidizing agent is hydrolizable into at least one oxidizing moiety having oxidizing properties.

151. The method of claim 150, further comprising wetting said treated region prior to said applying.

152. The method of claim 150, wherein said oxidizing agent comprises a chlorinated isocyanurate.

153. The method of claim 152, wherein said chlorinated isocyanurate is selected from the group consisting of trichloro(iso)cyanurate and sodium dichloro(iso)cyanurate.

154. The method of claim 152, wherein said at least one oxidizing moiety comprises free chlorine.

155. The method of claim 150, wherein said skin ailment is caused by a microorganism.

156. The method of claim 155, wherein said microorganism is selected from the group consisting of a virus, bacteria and a fungi.

157. The method of claim 150, wherein said skin ailment is caused by a human papilloma virus.

158. A method of preparing a pharmaceutical composition for treating skin or mucosal membranes aliments, the method comprising polymerizing a mixture of a silicone polymer and an oxidizing agent, so as to obtain said oxidizing agent entrapped within said silicone polymer formed upon polymerization.

159. The method of claim 158, further comprising polymerizing a second silicone polymer so as to obtain a second polymerized silicone polymer and filling said second polymerized silicone polymer with said mixture of said silicone polymer and said oxidizing agent.

160. A method of preparing a pharmaceutical composition for treating skin or mucosal membranes aliments, the method comprising polymerizing a silicone polymer so as to form a polymerized silicone polymer and loading said polymerized silicone polymer with an oxidizing agent, so as to obtain said oxidizing agent entrapped within said polymerized silicone polymer.

161. The method of claim 160, wherein said loading precedes said polymerizing.

162. The method of claim 160, wherein said polymerizing proceeds said loading.

163. A method of preparing a pharmaceutical composition for treating skin or mucosal membranes aliments, the method comprising polymerizing a silicone polymer and applying thereon an oxidizing agent.
